(19) United States
(12) Patent Application Publication
(10) Pub. No.: US 2016/0287621 A1
(43) Pub. Date: Oct. 6, 2016

(54) BIOLOGICAL PRODUCTION OF NAD PRECURSORS AND ANALOGS

(71) Applicant: PRESIDENT AND FELLOWS OF HARVARD COLLEGE, Cambridge, MA (US)

(72) Inventors: David A. Sinclair, Chestnut Hill, MA (US); Po Hien Ear, Brookline, MA (US)

(73) Assignee: President and Fellows of Harvard College, Cambridge, MA (US)

(21) Appl. No.: 15/034,953

(22) PCT Filed: Nov. 6, 2014
(86) PCT No.: PCT/US14/64287

§ 371 Date: May 6, 2016

Related U.S. Application Data
(60) Provisional application No. 61/900,962, filed on Nov. 6, 2013.

(51) Int. Cl. A61K 31/7076; C12N 15/81; A61K 31/519; C12P 19/32

(52) U.S. Cl. CPC A61K 31/7076; C12P 19/32; C12N 15/81; A61K 31/519; A23L 1/3018; A23L 1/3058; A23V 2002/00

(57) ABSTRACT

Aspects of the invention relate to methods for producing NAD precursors such as nicotinamide mononucleotide (NMN), comprising providing an isolated cell that overexpresses a nicotinamide phosphoribosyltransferase (Nampt) enzyme and culturing the cell in the presence of nicotinamide (NAM).

### RELATED APPLICATIONS

This application claims the benefit under 35 U.S.C. §119(e) of U.S. Provisional Application Ser. No. 61/900,962, entitled “Biological Production of NAD Precursors and Analogs”, filed on Nov. 6, 2013, which is herein incorporated by reference in its entirety.

### FIELD OF INVENTION

Aspects of the invention relate to production of nicotinamide mononucleotide (NMN) and related molecules through metabolic engineering.

### BACKGROUND OF INVENTION

Mammalian sirtuins (SIRT1-7) are a conserved family of NAD+-dependent lysine-modifying enzymes that modulate the physiological response to dietary changes. SIRT1, 6, and 7 are nuclear, SIRT2 is cytoplasmic and SIRT3-5 are mitochondrial. SIRT1 is the best studied sirtuin. As nuclear and cytoplasmic protein, SIRT1 sits at the nexus of key signaling pathways that affect cardiovascular health including Notch, AMPK, insulin/IGF-1 signaling, eNOS, mTOR, SREBP, PGC-1a, Hif1-a, FOXO1/3, low-density lipoprotein receptor (lox-1), LDL-R expression [2-6]. The expression of SIRT1 is elevated in a number of tissues following restriction of caloric intake by 30-40% (CR) [7].

### SUMMARY OF INVENTION

Aspects of the invention relate to methods for producing nicotinamide mononucleotide (NMN) as well as compositions such as cosmetics, pharmaceuticals and neutraceuticals that comprise NMN.

In some embodiments, methods comprise providing an isolated cell that overexpresses a nicotinamide phosphoribosyltransferase (Nampt) enzyme; and culturing the isolated cell in the presence of nicotinamide (NAM).

In some embodiments, the isolated cell has reduced expression and/or activity of one or more of Pnc1, Std1 and Ins1 relative to a wild type cell. In some embodiments, the isolated cell overexpresses a 5-phosphoribosyl-1-pyrophosphate (Prs1) enzyme. In some embodiments, the isolated cell recombinantly expresses a nucleic acid encoding the Nampt enzyme and/or a nucleic acid encoding the Prs1 enzyme.

In some embodiments, the isolated cell recombinantly expresses a nucleic acid encoding the Nampt enzyme and a nucleic acid encoding the Prs1 enzyme that are fused to each other. In some embodiments, adenine biosynthesis is disrupted in the isolated cell. In certain embodiments, the isolated cell has reduced expression of an adenine deaminase enzyme. In some embodiments, the isolated cell is cultured in the presence of adenine.

In some embodiments, expression of Nicotinamide Mononucleotide Adenylyltransferase 1 (NMA1) and/or Nicotinamide Mononucleotide Adenylyltransferase 2 (NMA2) is reduced in the isolated cell relative to a wild type cell.

In some embodiments, the NMN is harvested from the isolated cell or is collected from cell culture medium produced by culturing the isolated cell. In some embodiments, the isolated cell is cultured in the presence of tryptophan. In some embodiments, the isolated cell is a fungal cell, such as a yeast cell. In certain embodiments, the yeast cell is a Saccharomyces cell. In some embodiments, the isolated cell is a bacterial cell.

Aspects of the invention relate to an isolated cell that recombinantly expresses a nucleic acid encoding a nicotinamide phosphoribosyltransferase (Nampt) enzyme and that has reduced expression of Pnc1 relative to a wild type cell. In some embodiments, the isolated cell has reduced expression of one or both of Std1 and Ins1 relative to a wild type cell. In some embodiments, the isolated cell overexpresses a 5-phosphoribosyl-1-pyrophosphate (Prs1) enzyme. In some embodiments, the isolated cell recombinantly expresses a nucleic acid encoding the Nampt enzyme and/or a nucleic acid encoding the Prs1 enzyme. In some embodiments, the isolated cell recombinantly expresses a nucleic acid encoding the Nampt enzyme and a nucleic acid encoding the Prs1 enzyme that are fused to each other.

In some embodiments, adenine biosynthesis is disrupted in the cell. In certain embodiments, the isolated cell has reduced expression of an adenine deaminase enzyme. In some embodiments, the isolated cell is cultured in the presence of adenine.

In some embodiments, expression of Nicotinamide Mononucleotide Adenylyltransferase 1 (NMA1) and/or Nicotinamide Mononucleotide Adenylyltransferase 2 (NMA2) is reduced in the isolated cell relative to a wild type cell.

In some embodiments, NMN is produced by culturing one or more of any of the isolated cells described herein. In some embodiments, the isolated cell is a fungal cell, such as a yeast cell. In some embodiments, the yeast cell is a Saccharomyces cell. In some embodiments, the isolated cell is a bacterial cell.

Further aspects of the invention relate to cell extracts produced from any of the isolated cells described herein.

Further aspects of the invention relate to compositions comprising cell extracts produced from any of the isolated cells described herein, wherein the composition comprises NMN. In some embodiments, the extracts comprise NMN. In some embodiments the extracts comprise at least one other metabolite, for example folate and/or S-adenosyl-L-methionine (SAM). In some embodiments, the extracts comprise NMN and folate. In some embodiments, the extracts comprise NMN and SAM. In some embodiments, the extracts comprise NMN, folate and SAM. In some embodiments, the folate and/or SAM are added to a composition comprising NMN from cellular extracts or NMN in a purified form, to produce a composition comprising NMN and folate and/or SAM.

Further aspects of the invention relate to cosmetics, pharmaceuticals, nutraceuticals or food products produced by culturing any of the isolated cells described herein.

Further aspects of the invention relate to cosmetics, pharmaceuticals, nutraceuticals or food products comprising the cell extracts produced from any of the isolated cells described herein.

Further aspects of the invention relate to methods for producing nicotinamide mononucleotide (NMN), comprising: providing an isolated cell that overexpresses one or more of Nicotinamide Mononucleotide Adenylyltransferase 1 (NMA1), Nicotinamide Mononucleotide Adenylyltransferase 2 (NMA2), nicotinamide mononucleotide adenylyltransferase 1 (NMNAT1), nicotinamide mononucleotide adenylyltransferase 2 (NMNAT2) and nicotinamide mononucleotide adenylyltransferase 3 (NMNAT3); and culturing the isolated cell to produce NMN.

Further aspects of the invention relate to methods for producing nicotinamide mononucleotide (NMN) in vitro, comprising: incubating NAD+ in vitro with one or more of Nicotinamide Mononucleotide Adenylyltransferase 1 (NMA1), Nicotinamide Mononucleotide Adenylyltransferase 2 (NMA2), nicotinamide mononucleotide adenylyltransferase 1 (NMNAT1), nicotinamide mononucleotide adenylyltransferase 2 (NMNAT2) and nicotinamide mononucleotide adenylyltransferase 3 (NMNAT3); thereby producing NMN in vitro.

Further aspects of the invention relate to a composition comprising cell extracts from recombinant cells that produce NMN. In some embodiments, the recombinant cells are yeast cells, such as Saccharomyces cells. In some embodiments, the recombinant cells express a nicotinamide phosphoribosyltransferase (Nampt) enzyme.

Further aspects of the invention relate to cosmetics, pharmaceuticals, nutraceuticals or food products comprising any of the compositions described herein.

Further aspects of the invention relate to isolated yeast strains that express a nicotinamide phosphoribosyltransferase (Nampt) enzyme and that have reduced expression of Pnc1 relative to a wild type cell.

Further aspects of the invention relate to cosmetic, pharmaceutical, nutraceutical or food products described herein, wherein the cosmetic is a cream, serum or lotion. In some embodiments, the cream, serum or lotion contains NMN and one or more of folate and SAM.

Further aspects of the invention relate to a cosmetic, pharmaceutical, nutraceutical or food product that contains NMN and one or more of folate and SAM. In some embodiments, the cosmetic is a cream, serum or lotion. In some embodiments, the cosmetic or pharmaceutical is administered in an eye drop, occlusive tape or wrapping, patch, or in a formulation for systemic administration, including a pill form or a composition for injection. In some embodiments, any of the cosmetic, pharmaceutical, nutraceutical or food products described herein can be used for treatment or prevention of psoriasis or rheumatoid arthritis.

Further aspects of the invention relate to methods for treating or preventing psoriasis or rheumatoid arthritis in a subject in need thereof, comprising administering to the subject an effective amount of a composition comprising NMN. In some embodiments, the composition further comprises folate and/or S-adenosyl-L-methionine.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 provides a schematic of a non-limiting embodiment depicting metabolic engineering of nicotinamide mononucleotide (NMN) production in budding yeast. Overexpression of single or fusion proteins of human nicotinamide phosphoribosyltransferase (Nampt) and 5-phosphoribosyl-1-pyrophosphate (Prs1) in S. cerevisiae to produce nicotinamide mononucleotide (NMN) is shown. Fusing Prs1 to Nampt ensures that the PRPP, a substrate for Nampt, is locally available at high concentration. Yeast is cultured in medium supplemented with excess nicotinamide (Nam), another substrate of Nampt. The NAD+ salvage pathway is altered to obtain a high level of NMN when genes encoding Pnc1, Std1 and Ins1 are disrupted. Adenine biosynthesis, a consumer of PRPP can be disrupted by deleting ADE genes and supplying exogenous adenine. Nma1/2 enzymes convert NMN or nicotinic acid mononucleotide (NaMN) to NAD+. The NAD+ de novo synthesis pathway is responsible for converting tryptophan to NaMN. High levels of NaMN can be generated by overexpressing the genes for its synthesis from Tryptophan, and deleting NMA1 and 2.

FIG. 2 demonstrates NMN production and quantification in yeast strains. MATα (BY4742) yeast strain and MATαpnc1 deletion yeast strains were transformed with the p413GAL1 empty plasmid, mNAMPT or hNAMPT. Cells were grown in synthetic complete medium without histidine and with 2% raffinose. Protein expression was induced by adding 2% galactose. Twenty micrograms of the soluble yeast cell lysate was used for NMN quantification.

FIG. 3 demonstrates NMN production and quantification in yeast strains. MATα (BY4742) yeast strain and MATαnma1 deletion yeast strain were transformed with the p416GAL1 empty vector (EV), hNAMPT-tag or hNAMPT-PRS1. Cells were grown in synthetic complete medium without uracil, with 2% raffinose and 2 mM nicotinamide. Protein expression was induced by adding 2% galactose. The MATαnma1 deletion yeast strain overexpressing hNAMPT-tag fusion protein was cultured in a bioreactor in rich medium with 1% Yeast extract, 2% Peptone, 2% Galactose, 2 mM nicotinamide and 0.004% Adenine. For NMN quantification, NMN is converted to NAD⁺ by nicotinamide mononucleotide adenyltransferase 1 (NMNAT1). NAD⁺ is converted to NADH by alcohol dehydrogenase. NMN levels are shown.

FIG. 4 shows the process of generating yeast extract with NMN.

FIG. 5 shows the effect of NMN extract and pure NMN on cells. A and B) Human dermal fibroblasts (HDFa) were treated with various concentration of NMN extract or pure NMN from Sigma (St. Louis, Mo.) respectively for 24 hrs and NAD+ levels were determined.

FIG. 6 shows other metabolites in the partially purified NMN extract that have synergistic effects for raising NAD⁺ levels in combination with NMN. A) and B) Human dermal fibroblasts (HDFa) were treated with 50 μM of pure NMN and different concentration of Folate or S-adenosyl-L-methionine (SAM) respectively for 24 hrs and NAD⁺ levels were determined.

### DETAILED DESCRIPTION

Sirtuin activation is linked to many beneficial physiological effects and has potential uses in the pharmaceutical, nutraceutical, food, and cosmetic industries. The metabolite NAD+ is an essential co-substrate for the activity of all sirtuins. Raising NAD+ levels is a novel approach to activating all sirtuins. Administration of the NAD precursor, NMN, and related molecules may be able to slow the aging process, be useful in preventing and treating multiple age-related diseases, slow the external appearance of aging, and could be useful as a food supplement, cosmetic or nutraceutical, either in pure or extract form. However, currently, NMN is a high-cost metabolite that can only be purchased from a chemical supplier (Sigma) for approximately $US 1600 per gram. Described herein is an efficient strategy for generating NMN through microbial engineering.

In some aspects, the invention relates to the surprising discovery that NMN in cellular extracts is more stable or has a better efficacy than purified NMN, such as at elevated temperatures.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations of thereof herein, is meant to encompass the items listed thereafter and equivalents thereof as well as additional items.

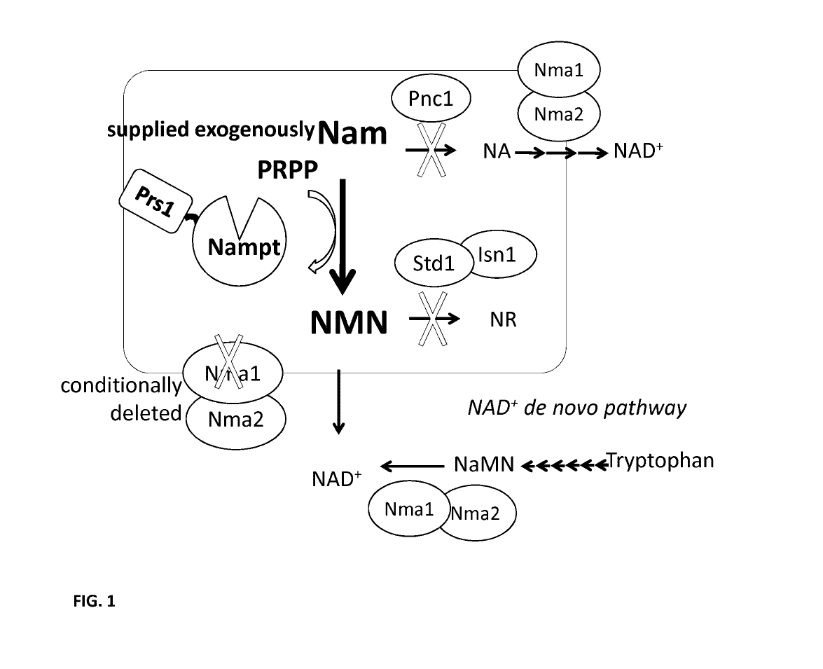

NAD+ (which may also be referred to herein as NAD) is an essential co-factor for several important enzymes (Canto and Auwerx, 2011). In mammals, NAD+ is generated from nicotinamide in a salvage pathway wherein nicotinamide phosphoribosyltransferase (NAMPT) converts nicotinamide to nicotinamide mononucleotide (NMN) which is then converted to NAD+ by nicotinamide mononucleotide adenylyltransferase (NMNAT) (FIG. 1) (Canto and Auwerx, 2011).

Yeast cells, such as S. cerevisiae cells, synthesize NAD from two pathways: the salvage pathway (via Pnc1) and de novo synthesis from the aromatic amino acid tryptophan. Both pathways feed into the salvage pathway. Nicotinic acid mononucleotide adenylyltransferases Nma1 and Nma2 catalyze the transfer of the adenylyl moiety of ATP to nicotinamide mononucleotide to form NAD (FIG. 1). Nma1 and Nma2 were named and characterized in 2002 (Anderson et al. (2002) J. Biol. Chem. 277(21):18881-90). In yeast, tryptophan is processed to make nicotinic acid mononucleotide (NaMN) but not NMN.

Aspects of the invention relate to methods of production of NMN and related molecules in microorganisms that do not regularly produce NMN through the pathway used by higher eukaryotes. The microorganism for production of NMN can be a yeast, such as the budding yeast, Saccharomyces cerevisiae, frequently used for metabolic engineering since it is easy and inexpensive to culture. Tools for manipulating the Saccharomyces cerevisiae genomic sequence and overexpressing exogenous proteins are well-known to one of ordinary skill in the art. It will be appreciated by one or ordinary skill in the art that other microorganisms, such as other fungal or bacterial microorganisms that are suitable for pharmaceutical, cosmetic, food or nutraceutical production are compatible with aspects of the invention.

In some aspects, methods and compositions associated with the invention involve overexpression of Nampt in cells, such as in S. cerevisiae cells, to convert the inexpensive precursor, nicotinamide (NAM), to NMN. In some embodiments, NAM is added in excess to the growth medium. To further ensure a high intracellular concentration of NAM, expression and/or activity of the nicotinamidase Pnc1 can be reduced in the cell. For example, the PNC1 gene can be mutated and/or deleted to reduce expression or activity of Pnc1. PNC1 has been characterized as part of the NAD salvage pathway (Anderson et al. (2002) J Biol Chem 277(21):18881-90; Anderson et al. (2003) Nature 423(6936):181-5).

PRPP can also be added to the culture medium. However, in contrast to NAM, PRPP is very costly and unstable (Zhang et al. (2011) Anal. Biochem. 412(1):18-25). Accordingly, in some embodiments, instead of adding PRPP to the culture medium, 5-phospho-ribosyl-1(alpha)-pyrophosphate synthetase (Prs1), an enzyme involved in PRPP synthesis, can be expressed at in the cells to ensure sufficient substrate for Nampt.

In addition to serving as a substrate for NMN biosynthesis, PRPP is commonly used to generate other nucleotides. To overcome the issue of PRPP being utilized by enzymes from other metabolic pathways, in some embodiments, a fusion protein of Nampt and Prs1 is generated (Nampt-Prs1) to promote the direct channeling of PRPP to Nampt to NMN. The PRPP produced from the Nampt-Prs1 chimeric protein is locally available for Nampt to use as substrate. It should be appreciated that the Nampt-Prs1 chimeric protein can be created by fusing the nucleic acid sequence encoding Nampt or a fragment thereof with the nucleic acid sequence of Prs1 or a fragment thereof.

Genes in the adenine biosynthetic pathway produce enzymes that are also PRPP consumers (e.g., ADE4) which in some embodiments are deleted. Adenine can be supplied exogenously to ensure the strain does not run out of adenine.

The Nma1/2 enzymes can convert NMN to NAD. To prevent or minimize this, in order to increase production of NMN, expression and/or activity of the Nma1/2 enzymes can be reduced. Since these enzymes are involved in NAD+ biogenesis, in some embodiments, it is preferable not to delete the genes encoding for both of these enzymes. In some embodiments, one or both of the genes can be conditionally targeted for degradation or deletion, or deleted individually. In some embodiments, if induced deletion or degradation is used, then the desired molecule, such as NMN, can be collected shortly after induction, such as within 10-60 minutes of induction.

According to further aspects of the invention, NMN production can be increased by preventing the hydrolysis of NMN to nicotinamide riboside (NR). This can be achieved by reducing expression and/or activity of the nucleotidases Sdt1 and Isn1. In budding yeast, these enzymes are not essential for cell survival. Deletion of the genes encoding these nucleotidases allows for increased production of NMN.

Yeast extracts from cells described herein can be used for nutraceutical or cosmetic use, optionally after a partial or a full purification or fractionation procedure. The increased stability and cost-effectiveness of NMN produced by methods described herein are advantageous relative to NMN produced by previous methods. In some embodiments, NMN that is produced by previously disclosed methods and is highly purified is less stable, such as at elevated temperatures, whereas NMN in cellular extracts has increased stability and efficacy, such as at higher temperatures.

In some embodiments, cellular extracts described herein comprise NMN. In some embodiments the extracts comprise at least one other metabolite, for example folate and/or S-adenosyl-L-methionine (SAM). In some embodiments, the extracts comprise NMN and folate. In some embodiments, the extracts comprise NMN and SAM. In some embodiments, the extracts comprise NMN, folate and SAM.

In some embodiments, folate and/or SAM are added to a composition comprising NMN from cellular extracts or purified NMN to produce a composition comprising NMN and folate and/or SAM. One of ordinary skill in the art would appreciate that the NMN, folate and/or SAM can be derived from any acceptable source. In some embodiments, the NMN is synthetic. In some embodiments, the NMN is naturally-produced and partially or fully purified. In some embodiments, the NMN is recombinantly produced. In some embodiments, the folate is synthetic. In some embodiments, the folate is naturally-produced and partially or fully purified. In some embodiments, the folate is folic acid. In some embodiments, the folate is recombinantly produced. In some embodiments, the SAM is synthetic. In some embodiments, the SAM is naturally-produced and partially or fully purified. In some embodiments, the SAM is recombinantly produced.

NMN can be harvested from cells or it can be secreted into the yeast medium and collected. Methods for NMN purification from cells are well-established in the art and are incorporated by reference in their entireties from: Armarego and Cha (2009) Purification of Laboratory Chemicals, 6^{th} Edition, page 690; Plaut and Plaut (1957) Biochemical Preparations, 5:56; Maplan and Stolzenbach (1957) Methods Enzymol 3:899; Kaplan et al. (1955) J Am Chem Soc, 77:815.

In another embodiment, NaMN (rather than NMN) can be produced at high levels by supplying tryptophan, overexpressing the rate limiting components of the NAD de novo pathway and deleting the NMA1/2 genes.

Aspects of the invention relate to production of the NAD+ precursor NMN, or a salt or prodrug thereof, which can be used in any embodiments in which increasing NAD+ levels would be beneficial.

In some embodiments, the expression and/or activity levels of one or more enzymes involved in NAD+ biosynthesis (de novo synthesis or salvage pathways) is altered. Enzymes involved in NAD+ biosynthesis such as nicotinate phosphoribosyl transferase 1 (NPT1), pyrazinamidase/nicotinamidase 1 (PNC1), nicotinic acid mononucleotide adenylyltransferase 1 (NMA1), nicotinic acid mononucleotide adenylyltransferase 2 (NMA2), nicotinamide N-methyltransferase (NNMT), nicotinamide phosphoribosyl transferase (NAMPT or NAMPRT), nicotinate/nicotinamide mononucleotide adenylyl transferase 1 (NMNAT-1), and nicotinamide mononucleotide adenylyl transferase 2 (NMNAT-2) are described in U.S. Pat. No. 7,977,049, which is incorporated by reference herein in its entirety.

Further aspects of the invention relate to overexpression of yeast NMA1 and/or NMA2 or mammalian NMNAT isoforms (NMNAT1,2,3) in cells associated with the invention, such as yeast cells, to convert endogenous NAD+ pools back to NMN.

Further aspects of the invention relate to methods for in vitro production of NMN, or a salt or prodrug thereof, involving incubating NAD+ in vitro with NMA1/2 or NMNAT isoforms to convert NAD+ to NMN in vitro.

It should be appreciated that NMN analogs may be useful for the treatment of diseases and cosmetic use. Cells associated with the invention that produce NMN can be engineered to add or delete groups to NMN or to make prodrugs. In addition, compounds produced can be converted to pro-drugs or new molecules by treatment with enzymes or chemicals in vitro after full or partial purification, as in the production of NADP, a related molecule, in U.S. Pat. No. 7,863,014 incorporated by reference herein in its entirety.

As used herein, the term “prodrug” means a derivative of a compound that can hydrolyze, oxidize, or otherwise react under biological conditions (in vitro or in vivo) to provide a compound described herein as useful in the methods of the invention. While prodrugs typically are designed to provide active compound upon reaction under biological conditions, prodrugs may have similar activity as a prodrug.

The references by Goodman and Gilman (The Pharmacological Basis of Therapeutics, 8th Ed, McGraw-Hill, Int. Ed. 1992, “Biotransformation of Drugs”, p 13-15); T. Higuchi and V. Stella (Pro-drugs as Novel Delivery Systems, Vol. 14 of the A.C.S. Symposium Series); and Bioreversible Carriers in Drug Design (E.B. Roche, ed., American Pharmaceutical Association and Pergamon Press, 1987) describing pro-drugs generally are hereby incorporated by reference. Prodrugs of the compounds described herein can be prepared by modifying functional groups present in said component in such a way that the modifications are cleaved, either in routine manipulation or in vivo, to the parent component. Typical examples of prodrugs are described for instance in WO 99/33795, WO 99/33815, WO 99/33793 and WO 99/33792, each of which is incorporated herein by reference for these teachings. Prodrugs can be characterized by increased bio-availability and are readily metabolized into the active inhibitors in vivo.

Examples of prodrugs include, but are not limited to, analogs or derivatives of the compounds described herein, further comprising biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of the compounds described herein that comprise —NO, —NO₂, —ONO, or —ONO₂ moieties. Prodrugs are prepared using methods known to those of skill in the art, such as those described by BURGER'S MEDICINAL CHEMISTRY AND DRUG DISCOVERY (1995) 172-178, 949-982 (Manfred E. Wolff ed., 5^{th} ed), the entire teachings of which are incorporated herein by reference.

As used herein, the terms “biohydrolyzable amide,” “biohydrolyzable ester,” “biohydrolyzable carbamate,” “biohydrolyzable carbonate,” “biohydrolyzable ureide” and “biohydrolyzable phosphate analogue” mean an amide, ester, carbamate, carbonate, ureide, or phosphate analogue, respectively, that either: 1) does not destroy the biological activity of the compound and confers upon that compound advantageous properties in vivo, such as uptake, duration of action, or onset of action; or 2) is itself biologically inactive but is converted in vivo to a biologically active compound. Examples of biohydrolyzable amides include, but are not limited to, lower alkyl amides, α-amino acid amides, alkoxyacyl amides, and alkylaminoalkylcarbonyl amides. Examples of biohydrolyzable esters include, but are not limited to, lower alkyl esters, alkoxyacyloxy esters, alkyl acylamino alkyl esters, and choline esters. Examples of biohydrolyzable carbamates include, but are not limited to, lower alkylamines, substituted ethylenediamines, aminoacids, hydroxyalkylamines, heterocyclic and heteroaromatic amines, and polyether amines.

Prodrugs can include fatty acids or lipids linked to the compounds described herein by the moieties described herein. Exemplary fatty acids include the omega-3 fatty acids eicosapentaenoic acid (EPA) and docosahexaenoic acid (DHA). Such prodrugs and the preparation thereof will be clear to the skilled person; reference is for instance made to the prodrug types and preparations described in U.S. Pat. No. 5,994,392, U.S. Pat. No. 4,933,324 and U.S. Pat. No. 5,284,876.

As used herein, the term “salt” or “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “solvate” includes any combination which may be formed by a compound of this invention with a suitable inorganic solvent (e.g. hydrates) or organic solvent, such as but not limited to alcohols, ketones, esters and the like. Such salts, hydrates, solvates, etc. and the preparation thereof will be clear to the skilled person; reference is for instance made to the salts, hydrates, solvates, etc. described in U.S. Pat. No. 6,372,778, U.S. Pat. No. 6,369,086, U.S. Pat. No. 6,369,087 and U.S. Pat. No. 6,372,733.

Aspects of the invention relate to compositions of matter including NAD+ precursors, such as NMN or a salt thereof or prodrug thereof. Further aspects of the invention relate to compositions of matter including an enzyme involved in NAD+ biosynthesis, such as NMNAT-1 or NAMPT, or an enzymatically active fragment thereof, or a nucleic acid encoding an enzyme involved in NAD+ biosynthesis, or an enzymatically active fragment thereof. In some embodiments, compositions include conjugates of agents described herein, such as fish oil conjugates.

Molecules produced using methods and compositions described herein, such as NMN or a salt or prodrug thereof, can be used for treatment and prevention of disorders. In some embodiments, the disorder is one which exhibits altered levels of NAD+ or NAD+ precursors, or one which could benefit from increased NAD+ biosynthesis. Examples of such disorders are disclosed in, and incorporated by reference from, U.S. Pat. Nos. 7,544,497, 8,017,634 and 8,114,626 and US Patent Publication Nos.: US2006/0025337 and US2008/0194803.

In some embodiments, the disorder is a lipid disorder. The lipid disorder can be an insulin resistance disorder. An “insulin resistance disorder,” as discussed herein, refers to any disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, hypercholesterolemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

In some embodiments, cells are treated in vitro to mimic caloric restriction, such as to extend their lifespan, e.g., to keep them proliferating longer and/or increasing their resistance to stress or prevent apoptosis. That compositions described herein may increase resistance to stress is based at least on the observation that Sir2 provides stress resistance and that PNC1 modulates Sir2 activity in response to cell stress (Anderson et al. (2003) Nature 423:181). This is particularly useful for primary cell cultures (i.e., cells obtained from an organism, e.g., a human), which are known to have only a limited lifespan in culture. Treating such cells according to methods described herein, e.g., by contacting them with an activating or lifespan extending composition will result in increasing the amount of time that the cells are kept alive in culture. Embryonic stem (ES) cells and pluripotent cells, and cells differentiated therefrom, can also be treated according to the methods described herein such as to keep the cells or progeny thereof in culture for longer periods of time. Primary cultures of cells, ES cells, pluripotent cells and progeny thereof can be used, e.g., to identify compositions having particular biological effects on the cells or for testing the toxicity of compositions on the cells (i.e., cytotoxicity assays). Such cells can also be used for transplantation into a subject, e.g., after ex vivo modification.

In some embodiments, compositions described herein may be used for extending the lifespan of a cell; extending the proliferative capacity of a cell; slowing ageing of a cell; promoting the survival of a cell; delaying cellular senescence in a cell; or mimicking the effects of calorie restriction.

In yet other embodiments, cells are treated in vivo, e.g., to increase their lifespan or prevent apoptosis. For example, skin can be protected from aging, e.g., developing wrinkles, by treating skin, e.g., epithelial cells, as described herein. In an exemplary embodiment, skin is contacted with a pharmaceutical or cosmetic composition comprising a composition described herein. Exemplary skin afflictions or skin conditions include disorders or diseases associated with or caused by inflammation, sun damage or natural aging. For example, the compositions find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dermatomyositis, skin cancer and the effects of natural aging.

Generally, compositions described herein may be used in methods for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the disease or condition does not result from oxidative stress. In certain embodiments, a method does not significantly increase the resistance of the subject to oxidative stress. In certain embodiments, the method does not act by decreasing the rate of occurrence of diseases that shorten the lifespan of a subject. In certain embodiments, a method does not act by reducing the lethality caused by a disease, such as cancer.

In yet another embodiment, compositions described herein can be administered to a subject, such as to generally increase the lifespan of its cells and to protect its cells against stress and/or against apoptosis. It is believed that treating a subject with a composition described herein is similar to subjecting the subject to hormesis, i.e., mild stress that is beneficial to organisms and may extend their lifespan. For example, a composition can be taken by subjects as a food or dietary supplement. In one embodiment, such a composition is a component of a multi-vitamin complex. Compositions can also be added to existing formulations that are taken on a daily basis, e.g., statins and aspirin. Compositions may also be used as food additives.

Compositions described herein could also be taken as one component of a multi-drug complex or as a supplement in addition to a multi-drug regimen. In one embodiment, this multi-drug complex or regimen would include drugs or compositions for the treatment or prevention of aging-related diseases, e.g., stroke, heart disease, arthritis, high blood pressure, Alzheimer's. In another embodiment, this multi-drug regimen would include chemotherapeutic drugs for the treatment of cancer. In a specific embodiment, a composition could be used to protect non-cancerous cells from the effects of chemotherapy.

Compositions described herein may be administered to a subject to prevent aging and aging-related consequences or diseases, such as stroke, heart disease, such as heart failure, arthritis, high blood pressure, and Alzheimer's disease. Other conditions that can be treated include ocular disorders, e.g., associated with the aging of the eye, such as cataracts, glaucoma, and macular degeneration. Compositions described herein can also be administered to subjects for treatment of diseases, e.g., chronic diseases, associated with cell death, such as to protect the cells from cell death. Exemplary diseases include those associated with neural cell death or muscular cell death, such as Parkinson's disease, Alzheimer's disease, multiple sclerosis, amniotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections.

Further non-limiting examples of disorders that could be treated or prevented include Parkinson's disease, Pick's disease, prion disease and other spongiform encephalopathies, diseases showing pathological aggregations of tau proteins, such as those that are associated with mutation of the tau gene on chromosome 17, such as dementia (including Lewy Body disease, mild cognitive impairment (MCI), Primary Senile Degenerative Dementia, Alzheimer Type Senile Dementia and Alzheimer Type Dementia), Parkinsonian disorders (including Lewy Body disease and Parkinsonism-linked to chromosome 17 (FTDP-17)), progressive supranuclear palsy (also known as Steele-Richardson-Olszewski Syndrome or Disease, Progressive Supranuclear Ophthalmoplegia), Pick's disease and corticobasal degeneration, diseases associated with expression or over-expression of p25, a toxic co-activator of cyclin-dependent kinase 5 (cdk5), such as Alzheimer's disease, or those that are associated with a mutation in the gene for superoxide dismutase 1 (SOD1G37R) and/or SOD1 aggregates encoded on chromosome 21q22.1, such as ALS. Other diseases include those that are associated with and/or caused by neuronal cell death caused by, e.g., a neurotoxic stress, such as disruption of calcium homeostasis and oxidative stress; and those in which neuronal cell death occurs as a result of a defect in cell cycle regulators, e.g., cdk5.

Other diseases that can be prevented or treated include those that relate to inflammation that results in cell death, e.g., neuronal cell death. Other diseases also include those associated with a trinucleotide repeat, such as Huntington's disease.

Cardiovascular diseases that can be treated or prevented include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to the retinal arterioles, the glomerular arterioles, the vasa nervorum, cardiac arterioles, and associated capillary beds of the eye, the kidney, the heart, and the central and peripheral nervous systems. The compositions may also be used for increasing HDL levels in plasma of an individual.

Yet other disorders that may be treated with compositions described herein include restenosis, e.g., following coronary intervention, and disorders relating to an abnormal level of high density and low density cholesterol. Compositions described herein may also be used for treating or preventing viral infections, such as infections by influenza, herpes or papilloma virus. They may also be used as antifungal agents, anti-inflammatory agents and neuroprotective agents.

Compositions described herein can also be administered to a subject suffering from an acute disease, e.g., damage to an organ or tissue, e.g., a subject suffering from stroke or myocardial infarction or a subject suffering from a spinal cord injury. Compositions can also be used to repair an alcoholic's liver.

Compositions described herein can also be administered to subjects who have recently received or are likely to receive a dose of radiation. In one embodiment, the dose of radiation is received as part of a work-related or medical procedure, e.g., working in a nuclear power plant, flying an airplane, an X-ray, CAT scan, or the administration of a radioactive dye for medical imaging; in such an embodiment, the composition is administered as a prophylactic measure. In another embodiment, the radiation exposure is received unintentionally, e.g., as a result of an industrial accident, terrorist act, or act of war involving radioactive material. In such a case, the composition is preferably administered as soon as possible after the exposure to inhibit apoptosis and the subsequent development of acute radiation syndrome.

Compositions described herein can be used to improve cognitive function. As used herein, “cognitive function” is understood to mean “of or pertaining to the mental processes of perception, memory, judgment and reasoning” (Random House Dictionary, Unabridged, 2nd ed., Random House, NY 1987). See also, Taber's Cyclopedic Medical Dictionary, F. A. Davison C. V., Philadelphia, 1989. The phrase “preventing deterioration of cognitive function” includes the prevention of deterioration of one of more of the following cognitive domains: orientation, attention and concentration, psychomotor speed and function, language and naming, verbal memory (immediate and delayed recall), category fluency, abstract reasoning and praxis (motor integration and executive control of complex learned movements). The prevention of deterioration of cognitive function can include prevention in patients not yet showing deterioration of cognitive function, and preferably, in patients who have shown deterioration in cognitive function.

One facet of cognitive function relates to learning and memory. The ability to learn relates a subject's capacity to acquire, retain or generalize specific skills or sets of information. A subject's ability to learn can be affected by deficiencies in attention, memory, perception or reasoning.

Methods and compositions described herein may be useful to a healthy subject of any age (e.g., children, adolescents, adults and the elderly). A healthy subject is a subject who is not known to have any deficits in cognitive performance relative to its usual cognitive performance, as can be tested with any of the tests further described herein. Thus, the methods described herein may be useful to improve a cognitive function, such as to stimulate or enhance memory, both short term and long term, and learning ability in subjects that suffer no deficits, chronic deficits or temporary/acute deficits. The methods may also be used to treat a subject having or at risk of having a condition or disease that impairs cognitive performance.

For example, the methods and compositions described herein may be used to improve cognitive performance of a healthy subject. Such a subject may simply desire to improve its learning skills, memory or attention span, for example.

Methods and compositions described herein may also be used to prevent or compensate for a cognitive impairment that results from a particular condition or state of a subject. For example, the methods may be used to prevent or repair the cognitive decline, such as memory loss, that is normally associated with aging. Thus, a subject that is noticing a cognitive impairment may benefit from the administration of a sirtuin activating agent. The methods may also apply to older subjects even if they have not noticed a cognitive impairment. A subject may be a subject that is at least about 40, 50, 60, 70 or 80 years old. Such a subject may wish to receive a sirtuin activating agent on a daily, weekly, or monthly basis, for example. A subject may also take a test to measure cognitive function and receive a sirtuin activating agent based on the results of the test.

Methods and compositions described herein may also be used to counteract factors that cause an impairment in cognitive performance in a subject, for example, sleep deprivation. Adequate sleep sustains cognitive performance, while less than adequate sleep leads to a decrease in cognitive performance over time as described by Thorne et al., Military Systems, Defense and Civil Institute of Environmental Medicine (1983); Newhouse et al., Neuropsychopharmacology 2: 153-164 (1989); Newhouse et al., Military Psychology 4: 207-233 (1992), all of which are incorporated herein by reference in their entirety.

The methods described herein may also be used to counteract the impairment of cognitive performance associated with or resulting from exposure to a condition or to a drug, such as a legal or FDA approved drug (medication or medicament) or an illegal or non-FDA approved drug. For example, a subject to be treated as described herein may be a subject having a substance-induced cognitive decline. A substance may be a drug, such as a barbiturate or benzodiazepine, cocaine, amphetamine; alcohol; or an anesthetic, such as a general anesthetic, e.g., halothane, isoflurane and fentanyl. A method may comprise administering to a subject a sirtuin activating agent prior to, during and/or after exposure to the drug. Thus, for example, to counteract the effect of alcohol on cognitive performance, a sirtuin activating agent may be taken before the ingestion of alcohol, at the same time, and/or after the ingestion of alcohol. The methods may also be used to counteract the effect of trauma, such as brain or head trauma, such as resulting from surgery (e.g., temporal lobe brain surgery) or an accident; brain masses caused by tumors or infection, herpes encephalitis and other brain infections; stroke; ischemia, such as transient ischemic attack (TIA); transient global amnesia; or electroconvulsive therapy.

The methods described herein may also be used to prevent or counteract cognitive impairment resulting from a condition or disease, e.g., a chronic condition or disease, or an age-related condition or disease. Exemplary diseases include neurodegenerative diseases and conditions of the central nervous system (CNS), such as Lewy body diseases, Alzheimer's disease, amyotrophic lateral sclerosis (ALS), Parkinson's Disease, Huntington's Chorea, senile dementia, Pick's disease, parkinsonism dementia syndrome, progressive subcortical gliosis, progressive supranuclear palsy, thalamic degeneration syndrome, hereditary aphasia, and myoclonus epilepsy.

Multiple sclerosis (MS), such as secondary or progressive MS, may also be treated, as well as its effects. Patients endure mild concentration or memory problems, while others experience depression, manic-depression, and paranoia. Other symptoms include sexual dysfunction, tremors, dizziness, slurred speech, trouble swallowing, urinary problems, and episodes of facial pain or inappropriate emotions.

Another condition that may be treated or prevented includes delirium. Delirium is a clinical state characterized by fluctuating disturbances in cognition, mood, attention, arousal, and self-awareness, which arises acutely, either without prior intellectual impairment or superimposed on chronic intellectual impairment (Merck Manual of Diagnosis and Therapy; www.merck.com). Delirium may be caused by a metabolic disease, a drug side effect, a structural lesion, or an infectious disease. For example, disorders causing delirium include anoxia, hyperkalemia, hyperparathyroidism, hyperthyroidism, hypoglycemia, hypokalemia, hypothyroidism, metabolic acidosis, postconcussion, postictal state, and transient ischemia. Drugs with anticholinergic properties such as antiemetics, antihistamines, antiparkinsonian drugs, antipsychotics, antispasmodics, muscle relaxants, tricyclic antidepressants may cause delirium. Other drugs that may cause delirium include alcohol, antihypertensives, benzodiazepines, cimetidine, digoxin, narcotics and other CNS depressants (www.merck.com).

Structural lesions that may cause delirium include vascular occlusion and cerebral infarction, subarachnoid hemorrhage, cerebral hemorrhage, primary or metastatic brain tumors, subdural hematomas, and brain abscesses. Most structural lesions can be detected by CT or MRI, and many produce focal neurologic signs observable during physical examination (www.merck.com).

Delirium may be caused by acute meningitis or encephalitis or by infections outside the brain, perhaps through the elaboration of toxins or production of fever. Pneumonia (even without impaired oxygenation), urinary tract infections, sepsis, or fever from viral infections can produce confusion in the vulnerable brain (www.merck.com).

Dementia is another condition that can be treated or prevented as described herein. Dementia is a chronic deterioration of intellectual function and other cognitive skills severe enough to interfere with the ability to perform activities of daily living (www.merck.com). Non-Alzheimer's dementias include Lewy body dementia, vascular dementia, Parkinson's disease, progressive supranuclear palsy, Huntington's disease (chorea), Pick's disease, frontal lobe dementia syndromes, normal-pressure hydrocephalus, subdural hematoma, Creutzfeld-Jakob disease, Gerstmann-Straussler-Scheinker disease (prion-related cause), general paresis and AIDS dementia. Other metabolic-toxic diseases causing dementia include anoxia, B12 deficiency, chronic drug-alcohol-nutritional abuse, folic acid deficiency, hypercalcemia associated with hyperparathyroidism, hypoglycemia, hypothyroidism, organ system failure, hepatic encephalopathy, respiratory encephalopathy, uremic encephalopathy and pellagra. Structural causes of dementia include Alzheimer's disease, ALS, brain trauma (acute severe), chronic subdural hematoma, dementia pugilistica, brain tumor, cerebellar degeneration, communicating hydrocephalus, Huntington's disease (chorea), irradiation to frontal lobes, MS, normal-pressure hydrocephalus, Parkinson's disease, Pick's disease, progressive multifocal leukoencephalopathy, progressive supranuclear palsy, surgery, vascular disease, multi-infarct dementia and Wilson's disease. Infectious causes of dementia include bacterial endocarditis, brain abscess, Creutzfeld-Jakob disease, Gerstmann-Straussler-Scheinker disease, HIV-related disorders, neurosyphilis (general paresis), tuberculous and fungal meningitis and viral encephalitis (www.merck.com).

Central nervous system disorders or diseases that may be treated include the following: (a) infectious Diseases of CNS: Tetanus, Poliomyelitis and other nonarthropod-borne viral disease, Creutzfeldt-Jakob disease, Rabies, Meningitis (Bacterial, Viral, Other), Encephalitis, Brain abscess; (b) Degenerative/Hereditary Diseases of CNS, such as Alzheimer disease, Parkinson disease, ALS, movement disorders, such as Periodic limb movement and Restless Legs Syndrome, Ataxia, Dystonia, Multiple system atrophies (e.g., Shy-Drager syndrome), Myoclonus, TICS (involuntary muscle contractions), periodic limb movement disorder (PLMD), Tourette's syndrome, Tremor (e.g., essential tremor, resting tremor) and Wilson disease; (c) Other Diseases of CNS: Mental retardation, Quadriplegia and other paralytic syndromes, Seizure disorders, Cerebral palsy; (d) Vascular Diseases of CNS: Cerebral hemorrhage, Intracranial hemorrhage, Transient cerebral ischemia, Cerebrovascular disease, Occlusion/stenosis of precerebral/cerebral arteries; (e) Neoplasms: Malignant intracranial neoplasm, Malignant neoplasm of nervous system; and (f) Ill-defined Symptoms referable to the Nervous System: Migraine, Headache, Hallucinations, Syncope and collapse, Dizziness/giddiness, Abnormal involuntary movement, Ataxia, Speech disturbance, and Sleep disorders.

Methods and compositions described herein may also be used to treat learning disabilities, such as attention deficit disorders, e.g., hyperactivity disorder (ADHD), such as those that occur in children. Accordingly, subjects in need of treatment may be subjects that are between 1 and 18 years old, between 1 and 10 years old, or between 1 and 5 years old.

Other conditions or diseases that can be treated include frontal-temporal dementia, mood and anxiety disorders, post-traumatic stress disorder, depression, Schizophrenia, autism, anxiety, Down syndrome, panic attacks, binge eating, social phobia, an affective disorder, a psychiatric disorder, mild cognitive impairment, cognitive complaint patients, seizures, neurodegenerative illnesses, dementia, head trauma or injury, hysteria accompanied by confusion, cognitive disorders; age-related dementias; age-induced memory impairment; ion deficit disorder; psychosis; cognitive deficits associated with psychosis; and drug-induced psychosis.

In other aspects, compositions described herein are used in food products, cosmetics or nutraceuticals. Use of fungal strains in production of food products and in fermentation is described in and incorporated by reference from U.S. Pat. No. 8,114,626.

Any form of cosmetic can be compatible with aspects of the invention. In some embodiments, the cosmetic is a cream, serum or lotion, including makeup, such as foundation, and sunscreen. In some embodiments, the cream, serum or lotion contains NMN. In some embodiments, the cream, serum or lotion contains NMN and one or more of folate and SAM. In some embodiments, the cosmetic or pharmaceutical is administered in an eye drop. For example, a cream, serum, lotion or eye drop can contain a composition comprising NMN. The composition can further comprise folate and/or SAM.

In some embodiments, the composition is for use in treating or preventing psoriasis or rheumatoid arthritis. In some embodiments, a cream, serum, lotion or eye drop contains NMN and folate, for treatment or prevention of psoriasis or rheumatoid arthritis. Aspects of the invention encompass methods of treating subjects in need thereof with effective amounts of compositions described herein.

In some embodiments, the composition is administered in an occlusive tape, wrapping, patch, or in a systemic medication (such as pills or compositions suitable for injection).

According to aspects of the invention, cell(s) that recombinantly express one or more genes associated with the invention, and the use of such cells in production of NMN, or a salt or prodrug thereof are provided. As one of ordinary skill in the art would be aware, homologous genes for these enzymes could be obtained from other species and could be identified by homology searches, for example through a protein BLAST search, available at the National Center for Biotechnology Information (NCBI) internet site (ncbi.nlm.nih.gov). Genes associated with the invention can be PCR amplified from DNA from any source of DNA which contains the given gene. In some embodiments, genes associated with the invention are synthetic. Any means of obtaining a gene encoding the enzymes associated with the invention are compatible with the instant invention.

The invention thus involves recombinant expression of genes encoding enzymes discussed above, functional modifications and variants of the foregoing, as well as uses relating thereto. Homologs and alleles of the nucleic acids associated with the invention can be identified by conventional techniques. Also encompassed by the invention are nucleic acids that hybridize under stringent conditions to the nucleic acids described herein. The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH2PO4 (pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of nucleic acids of the invention (e.g., by using lower stringency conditions). The skilled artisan also is familiar with the methodology for screening cells and libraries for expression of such molecules which then are routinely isolated, followed by isolation of the pertinent nucleic acid molecule and sequencing.

In general, homologs and alleles typically will share at least 75% nucleotide identity and/or at least 90% amino acid identity to the sequences of nucleic acids and polypeptides, respectively, in some instances will share at least 90% nucleotide identity and/or at least 95% amino acid identity and in still other instances will share at least 95% nucleotide identity and/or at least 99% amino acid identity. The homology can be calculated using various, publicly available software tools developed by NCBI (Bethesda, Maryland) that can be obtained through the NCBI internet site. Exemplary tools include the BLAST software, also available at the NCBI internet site (www.ncbi.nlm.nih.gov). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code. The invention also embraces codon optimization to suit optimal codon usage of a host cell.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as enzymatic activity. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

The invention embraces variants of polypeptides. As used herein, a “variant” of a polypeptide is a polypeptide which contains one or more modifications to the primary amino acid sequence of the polypeptide. Modifications which create a variant can be made to a polypeptide 1) to reduce or eliminate an activity of a polypeptide; 2) to enhance a property of a polypeptide; 3) to provide a novel activity or property to a polypeptide, such as addition of an antigenic epitope or addition of a detectable moiety; or 4) to provide equivalent or better binding between molecules (e.g., an enzymatic substrate). Modifications to a polypeptide are typically made to the nucleic acid which encodes the polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the amino acid sequence. One of skill in the art will be familiar with methods for predicting the effect on protein conformation of a change in protein sequence, and can thus “design” a variant of a polypeptide according to known methods. One example of such a method is described by Dahiyat and Mayo in Science 278:82 87, 1997, whereby proteins can be designed de novo. The method can be applied to a known protein to vary only a portion of the polypeptide sequence. By applying the computational methods of Dahiyat and Mayo, specific variants of a polypeptide can be proposed and tested to determine whether the variant retains a desired conformation.

In general, variants include polypeptides which are modified specifically to alter a feature of the polypeptide unrelated to its desired physiological activity. For example, cysteine residues can be substituted or deleted to prevent unwanted disulfide linkages. Similarly, certain amino acids can be changed to enhance expression of a polypeptide by eliminating proteolysis by proteases in an expression system (e.g., dibasic amino acid residues in yeast expression systems in which KEX2 protease activity is present).

Mutations of a nucleic acid which encode a polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a gene or cDNA clone to enhance expression of the polypeptide. The activity of variant polypeptides can be tested by cloning the gene encoding the variant polypeptide into a bacterial or mammalian expression vector, introducing the vector into an appropriate host cell, expressing the variant polypeptide, and testing for a functional capability of the polypeptides as disclosed herein.

The skilled artisan will also realize that conservative amino acid substitutions may be made in polypeptides to provide functionally equivalent variants of the foregoing polypeptides, i.e., the variants retain the functional capabilities of the polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Exemplary functionally equivalent variants of polypeptides include conservative amino acid substitutions in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In general, it is preferred that fewer than all of the amino acids are changed when preparing variant polypeptides. Where particular amino acid residues are known to confer function, such amino acids will not be replaced, or alternatively, will be replaced by conservative amino acid substitutions. Preferably, 1, 2, 3, 4, 5, 6, 7, 8, 9, 10, 11, 12, 13, 14, 15, 16, 17, 18, 19, 20 residues can be changed when preparing variant polypeptides. It is generally preferred that the fewest number of substitutions is made. Thus, one method for generating variant polypeptides is to substitute all other amino acids for a particular single amino acid, then assay activity of the variant, then repeat the process with one or more of the polypeptides having the best activity.

Conservative amino-acid substitutions in the amino acid sequence of a polypeptide to produce functionally equivalent variants of the polypeptide typically are made by alteration of a nucleic acid encoding the polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a polypeptide.

The invention encompasses any type of cell that recombinantly expresses genes associated with the invention, including prokaryotic and eukaryotic cells. In some embodiments the cell is a bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Agrobacterium spp. and Pantoea spp. The bacterial cell can be a Gram-negative cell such as an Escherichia coli (E. coli) cell, or a Gram-positive cell such as a species of Bacillus.

In other embodiments the cell is a fungal cell such as yeast cells, e.g., Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp. and industrial polyploid yeast strains. Preferably the yeast strain is a S. cerevisiae strain. Other examples of fungi include Aspergillus spp., Pennicilium spp., Fusarium spp., Rhizopus spp., Acremonium spp., Neurospora spp., Sordaria spp., Magnaporthe spp., Allomyces spp., Ustilago spp., Botrytis spp., and Trichoderma spp. In other embodiments the cell is an algal cell, a plant cell, or a mammalian cell. It should be appreciated that some cells compatible with the invention may express an endogenous copy of one or more of the genes associated with the invention as well as a recombinant copy. In some embodiments if a cell has an endogenous copy of one or more of the genes associated with the invention then the methods will not necessarily require adding a recombinant copy of the gene(s) that are endogenously expressed. In some embodiments the cell may endogenously express one or more enzymes from the pathways described herein and may recombinantly express one or more other enzymes from the pathways described herein for efficient production of NMN.

In some embodiments, one or more of the genes associated with the invention is expressed in a recombinant expression vector. As used herein, a “vector” may be any of a number of nucleic acids into which a desired sequence or sequences may be inserted by restriction and ligation for transport between different genetic environments or for expression in a host cell. Vectors are typically composed of DNA although RNA vectors are also available. Vectors include, but are not limited to: plasmids, fosmids, phagemids, virus genomes and artificial chromosomes.

A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., (β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the enzymes of the claimed invention is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Fourth Edition, Cold Spring Harbor Laboratory Press, 2012. Cells are genetically engineered by the introduction into the cells of heterologous DNA (RNA). That heterologous DNA (RNA) is placed under operable control of transcriptional elements to permit the expression of the heterologous DNA in the host cell. Heterologous expression of genes associated with the invention, for production NMN, is demonstrated in the Examples section using yeast cells. As one of ordinary skill in the art would appreciate, the novel method for producing NMN can also be expressed in other cells, including other fungal cells, bacterial cells, archaeal cells, mammalian cells, plant cells, etc.

Nucleic acid molecules encoding enzymes of the claimed invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

In some embodiments one or more genes associated with the invention is expressed recombinantly in a fungal or bacterial cell. Fungal or bacterial cells according to the invention can be cultured in media of any type (rich or minimal) and any composition. As would be understood by one of ordinary skill in the art, routine optimization would allow for use of a variety of types of media. The selected medium can be supplemented with various additional components. Some non-limiting examples of supplemental components include carbon sources, such as glucose, galactose and raffinose, antibiotics, IPTG for gene induction, and ATCC Trace Mineral Supplement. Similarly, other aspects of the medium, and growth conditions of the cells of the invention may be optimized through routine experimentation. For example, pH and temperature are non-limiting examples of factors which can be optimized. In some embodiments, factors such as choice of media, media supplements, and temperature can influence production levels of NMN. In some embodiments the concentration and amount of a supplemental component may be optimized. In some embodiments, how often the media is supplemented with one or more supplemental components, and the amount of time that the media is cultured before harvesting NMN, or a salt or prodrug thereof, is optimized. In some embodiments, the cells are grown in rich media. In some embodiments, the cells are grown in the presence of galactose, nicotinamide and/or adenine. In some embodiments, the pH is about 5. In some embodiments, the pH is between 4 and 6, or between 4.5 and 5.5. For example, in some embodiments, the pH is about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9 or 6.0. In other embodiments, the pH is lower than 4 or greater than 6.

Liquid cultures used to grow cells associated with the invention can be housed in any of the culture vessels known and used in the art. In some embodiments large scale production in an aerated reaction vessel such as a stirred tank reactor or bioreactor can be used to produce large quantities of NMN, or a salt or prodrug thereof. Methods of culturing fungal strains and recovery of a product from culturing of fungal strains is described further and incorporated by reference from U.S. Pat. No. 8,114,626.

In some embodiments, a titer of approximately 5 mg of NMN extract per gram of soluble yeast protein after filtration is produced. In some embodiments, a titer of approximately 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0. 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9. 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50 mg of NMN per gram of soluble yeast protein after filtration is produced.

Aspects of the invention include strategies to optimize production of NMN, or a salt or prodrug thereof, from a cell. Optimized production of NMN, or a salt or prodrug thereof, refers to producing a higher amount of NMN, or a salt or prodrug thereof, following pursuit of an optimization strategy than would be achieved in the absence of such a strategy. One strategy for optimization is to increase expression levels of one or more genes associated with the invention through selection of appropriate promoters and/or ribosome binding sites. In some embodiments, this may include the selection of high-copy number plasmids, or low or medium-copy number plasmids. In some embodiments, the plasmid is medium-copy number plasmid such as pETDuet. The step of transcription termination can also be targeted for regulation of gene expression, through the introduction or elimination of structure such as stem-loops.

In some embodiments, it may be advantageous to use a cell that has been optimized for production of NMN, or a salt or prodrug thereof. In some embodiments, screening for one or more mutations that lead to enhanced production of NMN, or a salt or prodrug thereof may be conducted through random mutagenesis, or through screening of known mutations. In some embodiments, shotgun cloning of genomic fragments can be used to identify genomic regions that lead to an increase in production of NMN, or a salt or prodrug thereof, through screening cells or organisms that have these fragments for production of NMN, or a salt or prodrug thereof. In some cases on or more mutation may be combined in the same cell or organism. In other embodiments, a wild-type cell can be used.

Optimization of production of NMN, or a salt or prodrug thereof can involve optimizing selection of fungal or bacterial strains for expression of recombinant pathways described herein. In some embodiments, use of a strain that is wild-type or close to wild-type, meaning a strain that has not been substantially genetically modified, may lead to increased titers of NMN, or a salt or prodrug thereof.

Optimization of protein expression may also require in some embodiments that a gene encoding an enzyme be modified before being introduced into a cell such through codon optimization for expression in a bacterial cell. Codon usages for a variety of organisms can be accessed in the Codon Usage Database (kasusa.or.jp/codon/).

In some embodiments, protein engineering can be used to optimize expression or activity of one or more enzymes associated with the invention. In certain embodiments a protein engineering approach could include determining the 3D structure of an enzyme or constructing a 3D homology model for the enzyme based on the structure of a related protein. Based on 3D models, mutations in an enzyme can be constructed and incorporated into a cell or organism, which could then be screened for an increase production of NMN, or a salt or prodrug thereof. In some embodiments, production of NMN, or a salt or prodrug thereof in a cell could be increased through manipulation of enzymes that act in the same pathway as the enzymes associated with the invention. For example in some embodiments it may be advantageous to increase expression of an enzyme or other factor that acts upstream of a target enzyme such as an enzyme associated with the invention. This could be achieved by over-expression of the upstream factor using any standard method.

Aspects of the invention relate to production of molecules such as NMN, or a salt or prodrug thereof, that can be administered to a subject. As used herein, the term “subject” refers to a human or non-human mammal. Non-human mammals include livestock animals, companion animals, laboratory animals, and non-human primates. Non-human subjects also specifically include, without limitation, chickens, horses, cows, pigs, goats, dogs, cats, guinea pigs, hamsters, mink, and rabbits. In some embodiments the subject is a patient. As used herein, a “patient” refers to a subject who is under the care of a physician, dentist, or other health care worker, including someone who has consulted with, received advice from or received a prescription or other recommendation from a physician or other health care worker. A patient is typically a subject having or at risk of having a disorder that exhibits altered levels of NAD+ or NAD+ precursors or could benefit from increased NAD+ biosynthesis by treatment with NMN.

As used herein, the term treat, treated, or treating when used with respect to an disorder refers to a prophylactic treatment which increases the resistance of a subject to development of the disease or, in other words, decreases the likelihood that the subject will develop the disease as well as a treatment after the subject has developed the disease in order to fight the disease or prevent the disease from becoming worse.

The term “effective amount” of an agent of the invention refers to the amount necessary or sufficient to realize a desired biologic effect. For example, an effective amount of an agent for treating a disorder is that amount sufficient to prevent an increase in one or more symptoms of a disorder in the subject or that amount necessary to decrease one or more symptoms of a disorder in the subject that would otherwise occur in the absence of the agent. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular composition being administered, the size of the subject, or the severity of the disease or condition. One of ordinary skill in the art can empirically determine the effective amount of a particular composition of the invention without necessitating undue experimentation.

The agents of the invention may be delivered to the subject on an as needed or desired basis. For instance a subject may self-administer the agents as desired or a physician or veterinarian may administer the agents. Additionally a physician, veterinarian or other health care worker may select a delivery schedule. In other embodiments of the invention, the agents are administered on a routine schedule. A “routine schedule” as used herein, refers to a predetermined designated period of time. The routine schedule may encompass periods of time which are identical or which differ in length, as long as the schedule is predetermined. For instance, the routine schedule may involve administration of the composition on a daily basis, multiple times a day, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve, for example, administration of the composition on a daily basis for the first week, followed by a monthly basis for several months, and then every three months after that. Any particular combination would be covered by the routine schedule as long as it is determined ahead of time that the appropriate schedule involves administration on a certain day.

The agents may be administered alone or in any appropriate pharmaceutical carrier, such as a liquid, for example saline, or a powder, for administration in vivo. They can also be co-delivered with larger carrier particle or within administration devices. The agents may be formulated. The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the agents can be administered to a subject by any mode. Administering a pharmaceutical composition of the present invention may be accomplished by any means known to the skilled artisan. Routes of administration include but are not limited to topical, oral, parenteral, intramuscular, intravenous, subcutaneous, mucosal, intranasal, sublingual, intratracheal, inhalation, ocular, vaginal, dermal, rectal, and by direct injection.

It is well known to those skilled in the art that agents may be administered to patients using a full range of routes of administration. As an example, agents may be blended with direct compression or wet compression tableting excipients using standard formulation methods. The resulting granulated masses may then be compressed in molds or dies to form tablets and subsequently administered via the oral route of administration. Alternately particle granulates may be extruded, spheronized and administered orally as the contents of capsules and caplets. Tablets, capsules and caplets may be film coated to alter dissolution of the delivery system (enteric coating) or target delivery of the particle to different regions of the gastrointestinal tract. Additionally, particles may be orally administered as suspensions in aqueous fluids or sugar solutions (syrups) or hydroalcoholic solutions (elixirs) or oils. The particles may also be administered directly by the oral route without any further processing.

The agents of the invention may be systemically administered in combination with a pharmaceutically acceptable vehicle such as an inert diluent or an assimilable edible carrier. They may be enclosed in hard or soft shell gelatin capsules or compressed into tablets. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of an active compound, e.g., calcium. The percentage of the compositions and preparations may, of course, be varied and may conveniently be between about 2 to about 60% of the weight of a given unit dosage form. The amount of active compound in such therapeutically useful compositions is such that an effective dosage level will be obtained. In some embodiments, agents described herein, such as NMN or a salt or prodrug thereof, are administered at a dosage of 250 mg-5 grams per day, by an oral route.

In some embodiments, the dose of NMN (in cellular extracts and/or in partially or fully purified form) administered to a subject ranges from 200 mg/Kg to 500 mg/Kg. In some embodiments, the concentration of NMN (in cellular extracts and/or in partially or fully purified form) administered to a subject ranges from about 1 μM to about 1 mM. For example, in some embodiments, the concentration of NMN (in cellular extracts and/or in partially or fully purified form) administered to a subject ranges from about 10 μM to about 900 μM, about 50 μM to about 800 μM, about 100 μM to about 700 μM, or about 200 μM to about 500 μM, including all intermediate values. In other embodiments, the concentration of NMN (in cellular extracts and/or in partially or fully purified form) administered to a subject is less than 1 μM or more than 1 mM. In some embodiments, a composition comprising NMN (in cellular extracts and/or in partially or fully purified form) is administered in an effective amount to a subject in need thereof.

In some embodiments, the composition comprising NMN (in cellular extracts and/or in partially or fully purified form) further comprises folate and/or S-adenosyl-L-methionine (SAM). In some embodiments, the folate is folic acid. In some embodiments, the concentration of folate ranges from about 1 μM to about 1 mM. In some embodiments, the concentration of folate ranges from about 10 μM to about 900 μM, about 20 μM to about 800 μM, about 30 μM to about 700 μM, about 40 μM to about 600 μM, or about 50 μM to about 500 μM, including all intermediate values. In other embodiments, the concentration of folate administered to a subject is less than 1 μM or more than 1 mM.

In some embodiments, the concentration of SAM ranges from about 1 μM to about 1 mM. In some embodiments, the concentration of SAM ranges from about 10 μM to about 900 μM, about 20 μM to about 800 μM, about 30 μM to about 700 μM, 40 μM to about 600 μM or about 50 μM to about 500 μM, including all intermediate values. In other embodiments, the concentration of SAM administered to a subject is less than 1 μM or more than 1 mM.

It should be appreciated that any concentration of NMN (in cellular extracts and/or in partially or fully purified form) can be combined with any concentration of folate and/or any concentration of SAM.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed.

To ensure full gastric resistance a coating impermeable to at least pH 5.0 can be helpful. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic, e.g., powder; for liquid forms, a soft gelatin shell may be used. The shell material of cachets could be thick starch or other edible paper. For pills, lozenges, molded tablets or tablet triturates, moist massing techniques can be used.

The agents of the invention may also be administered orally as a nutraceutical additive. As used herein, “nutraceutical” can refer to a range of products including nutritional supplements, health foods and additives. Yeast extracts are a common ingredient used to impart savory taste in commercially processed foods. Non-limiting examples of foods containing yeast extracts include frozen meals, crackers, gravy, stock, cheese, yogurt, beer and wine. In some embodiments, the yeast extracts comprising NMN described herein are used as nutraceutical additives in food products.

The agents of the invention may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In some embodiments the compositions of the invention are not encapsulated or formulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

For topical administration, the agents of the invention will generally be administered as compositions or formulations, in combination with a dermatologically acceptable carrier, which may be a solid or a liquid. Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In some embodiments, the cellular extracts or compositions are administered as a cosmetic. In some embodiments, the cosmetic is a skin cream, serum or lotion. Skin creams or lotions typically comprise from about 5% to about 98% water, 1% to 85% oil and from about 0.1% to 20% of one or more surfactants. Preferably the surfactants are nonionic and may be in the form of silicones or organic nonionic surfactants. The cell extracts or compositions may be found in the water or oil phase of the skin cream, serum or lotion depending upon the solubility of the cell extract or composition. Cosmetic compositions are described further in, and specifically incorporated by reference from, US Patent Publication No. 2009/0035237.

The compositions of the inventions may include a physiologically or pharmaceutically acceptable carrier, excipient, or stabilizer mixed with the particles. The term “pharmaceutically acceptable” means a non-toxic material that does not interfere with the effectiveness of the biological activity of the active ingredients. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency. A pharmaceutical preparation is a composition suitable for administration to a subject. Such preparations are usually sterile and prepared according to GMP standards, particularly if they are to be used in human subjects. In general, a pharmaceutical composition or preparation comprises the particles, and optionally agents of the invention and a pharmaceutically-acceptable carrier, wherein the agents are generally provided in effective amounts.

Agents may also be suspended in non-viscous fluids and nebulized or atomized for administration of the dosage form to nasal membranes. Agents may also be delivered parenterally by either intravenous, subcutaneous, intramuscular, intrathecal, intravitreal or intradermal routes as sterile suspensions in isotonic fluids. In some embodiments compositions described herein are administered in an eye drop.

Finally, agents may be nebulized and delivered as dry powders in metered-dose inhalers for purposes of inhalation delivery. For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of for use in an inhaler or insufflator may be formulated containing the microparticle and optionally a suitable base such as lactose or starch. Those of skill in the art can readily determine the various parameters and conditions for producing aerosols without resort to undue experimentation. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. Techniques for preparing aerosol delivery systems are well known to those of skill in the art. Generally, such systems should utilize components which will not significantly impair the biological properties of the agent in the nanoparticle or microparticle (see, for example, Sciarra and Cutie, “Aerosols,” in Remington's Pharmaceutical Sciences, 18th edition, 1990, pp. 1694-1712; incorporated by reference).

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Massachusetts.

Agents, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Further aspects of the invention relate to kits comprising a pharmaceutical composition comprising a therapeutically effective amount of NMN or a salt or prodrug thereof and instructions for administration of the pharmaceutical composition. In some aspects of the invention, the kit can include a pharmaceutical preparation vial, a pharmaceutical preparation diluent vial, and the agent(s). The diluent vial can contain a diluent such as physiological saline for diluting what could be a concentrated solution or lyophilized powder of the agent of the invention. In some embodiments, the instructions include instructions for mixing a particular amount of the diluent with a particular amount of the concentrated pharmaceutical preparation, whereby a final formulation for injection or infusion is prepared. In some embodiments, the instructions include instructions for use in a syringe or other administration device. In some embodiments, the instructions include instructions for treating a patient with an effective amount of an agent. It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1

### NMN Biosynthesis in Yeast and Quantification in Yeast Cell Lysate using NMNAT Assay

Sirtuins can protect against numerous age-related diseases, including cancer, cardiovascular diseases, neurodegeneration, inflammatory disorders (e.g. COPD, IBD, psoriasis), type 1 and 2 diabetes, and ischemia-reperfusion injury [1]. They also play key roles in regulating circadian rhythms and even neurological disorders (e.g. anxiety). The expression of SIRT1 is elevated in a number of tissues following restriction of caloric intake by 30-40% (CR) [7], one intervention generally accepted to extend lifespan. Overexpression or pharmacological activation of SIRT1 reproduces many of the health benefits of CR, including protection from metabolic decline [4-6, 8-10], cardiovascular disease [11], cancer [12, 13] and neurodegeneration [14-16]. Numerous studies have linked the health benefits of CR to increased mitochondrial biogenesis [17-20] and delayed mitochondrial decline [21].

Mouse genetic models to study SIRT1 have demonstrated that Sirt1 suppresses inflammation, increases antioxidant activity, [22] and protects mice from vascular smooth muscle hypertrophy and cardiomyopathies [23], antagonizes arterial calcification [Takemura, 2011], and promotes new blood vessel growth both in zebrafish and in mice by regulating notch signaling [24, 25]. The SIRT1 activating molecule resveratrol [26] lowers protects against the negative effects of aging and a high fat diet on atherosclerosis, endothelial function, insulin sensitivity, mitochondrial function, and lifespan, among other effects [2-6]. A recent small clinical trial showed that the SIRT1-specific activator SRT2104 improved the serum lipid profile of cigarette smokers, including a reduction in total cholesterol, LDL and triglycerides (Venkatasubramanian et al. (2013) J Am Heart Assoc Jun 14;2(3):e000042).

### Raising NAD⁺ Levels—A Novel Approach to Activating all Sirtuins

While specific SIRT1 activators may prove efficacious in treating age-related diseases,¹⁰ there is increasing evidence that pan-sirtuin activators may be more effective.^{11,12} The metabolite NAD⁺ is an essential co-substrate for the activity of all sirtuins. Levels of NAD⁺ decline in response to a high-fat diet, DNA damage and aging.¹³ Reports on sirtuin-dependent dependent beneficial effects of increasing NAD⁺ levels¹¹⁻¹³ suggest that NMN and related molecules may be useful in treating and preventing diseases associated with ageing and as a food supplement, cosmetic or nutraceutical. Described herein is the microbial production of NMN through metabolic engineering.

### Methods and Materials

### Yeast Strains and Protein Extraction

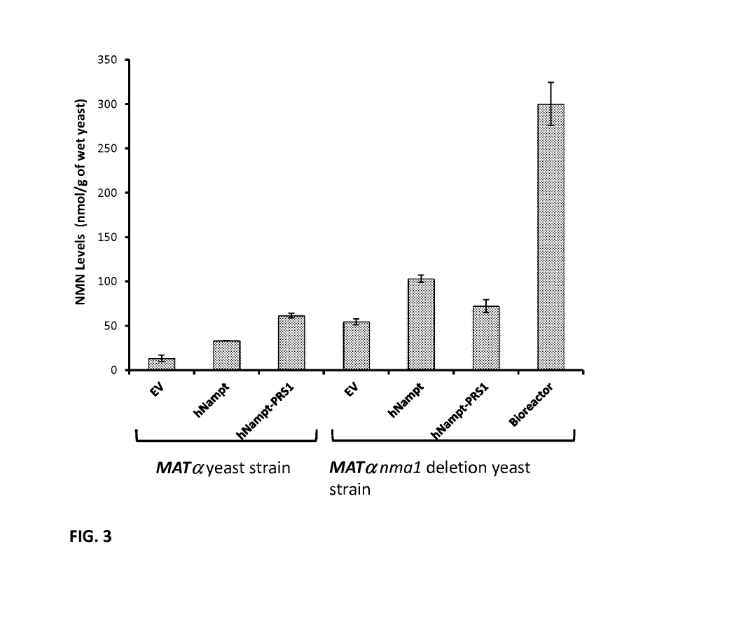

In the experiments depicted in FIG. 3, the MATα and MATα (BY4741 and BY4742) yeast strain and MATα and MATα pnc1, std1, isn1, nma1 deletion single deletion strains and double deletion strains (pnc1std1; pnc1nma1; isn1std1 and isn1nma1) were transformed with the p413GAL1 empty plasmid, p413GAL1-mouse or human NAMPT-tag (a fragment of the FCY1 gene) or, p413GAL1-human NAMPT-PRS1, p416GAL1 empty plasmid, p416GAL1-human NAMPT, p416GAL1-mouse or human NAMPT-tag (a fragment of the FCY1 gene) or, p416GAL1-human NAMPT-PRS1. A colony was inoculated and grown overnight in synthetic complete medium without histidine (for p413Gal1 plasmids) or uracil (for p416Gal1 plasmids) and with 2% raffinose. Nicotinamide (2 mM) was also added to the medium to serve as a substrate for Nampt. Galactose was added to the culture medium to a final concentration of 2% to induce the expression of the fusion genes (human NAMPT-tag and human NAMPT-PRS1). Yeast cells were collected, resuspended in RIPA buffer with protease inhibitors and lysed by bead beating. The soluble fraction was collected for nicotinamide mononucleotide (NMN) quantification.

For culture in the bioreactor, the MATα nma1 strain overexpressing human NAMPT-tag gene was cultured in 25 mL of synthetic complete medium without uracil and with 2% raffinose for 16 hours. This culture was transferred to a flask containing 200 mL of synthetic complete medium without uracil and with 2% galactose and grown for 16 hours. Finally, the yeast culture was transferred to the bioreactor containing 4 L of rich medium containing 1% yeast extract, 2% peptone, 2% galactose, 2 mM nicotinamide and 0.004% adenine and allow to grow for 24 hours.

Yeast cells were collected and stored at −80 ° C. For NMN extraction, yeast cells were washed with distilled water and resuspended in 10 mM Tris buffer at pH 5.0 and lysed by bead beating. The soluble fraction was collected, filtered through a 10 KDa cutoff membrane and ready for nicotinamide mononucleotide (NMN) quantification.

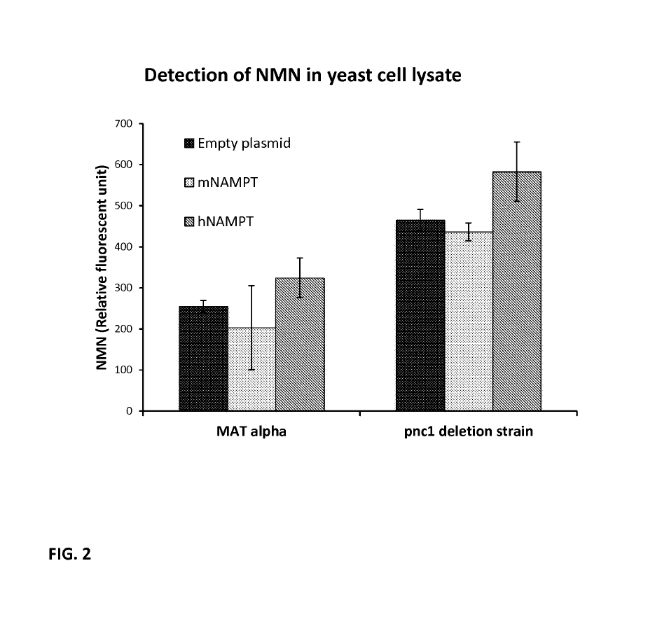

In the experiments depicted in FIG. 2, The MATα (BY4742) yeast strain and MATα (BY4741) pnc1 deletion strain were transformed with the p413GAL1 empty plasmid, p413GAL1-mouse NAMPT or p413GAL1-human NAMPT. A colony was inoculated and grown overnight in synthetic complete medium without histidine and with 2% raffinose. Nicotinamide (2 mM) was also added to the medium to serve as a substrate for Nampt. Galactose was added to the culture medium to a final concentration of 2% to induce the expression of the mouse and human NAMPT. Yeast cells were collected, resuspended in RIPA buffer with protease inhibitors and lysed by bead beating. The soluble fraction was collected for nicotinamide mononucleotide (NMN) quantification.

### NMN Quantification

Nicotinamide mononucleotide (NMN) can be detected by using the mouse NAD synthase nicotinamide mononucleotide adenylyltransferase (mNMNAT1) and the yeast alcohol dehydrogenase (yAD) to convert NMN to nicotinamide adenine dinucleotide (NAD⁺ and NADH). The final product of this assay is NADH. In brief, twenty micrograms of the yeast soluble protein was used for NMN quantification in a Hepes buffer containing mNMNAT1 and yAD. As a control, twenty micrograms of the same yeast soluble protein was used in a parallel reaction in a Hepes buffer without mNMNAT1. Samples were incubated at 37° C. for 15 minutes, stopped by adding EDTA, and NADH was quantified by excitation at 340 nm and emission at 460 nm. The overall NMN was calculated by subtracting the amount of NADH in the enzymatic reaction without mNMNAT1 to the amount of NADH in the enzymatic reaction with mNMNAT1.

FIG. 2 demonstrates NMN production and quantification in yeast strains MATα (BY4742) and MATαpnc1 using methods described above. FIG. 3 demonstrates NMN production and quantification in yeast strains MATα and MATα nma1 using methods described above.

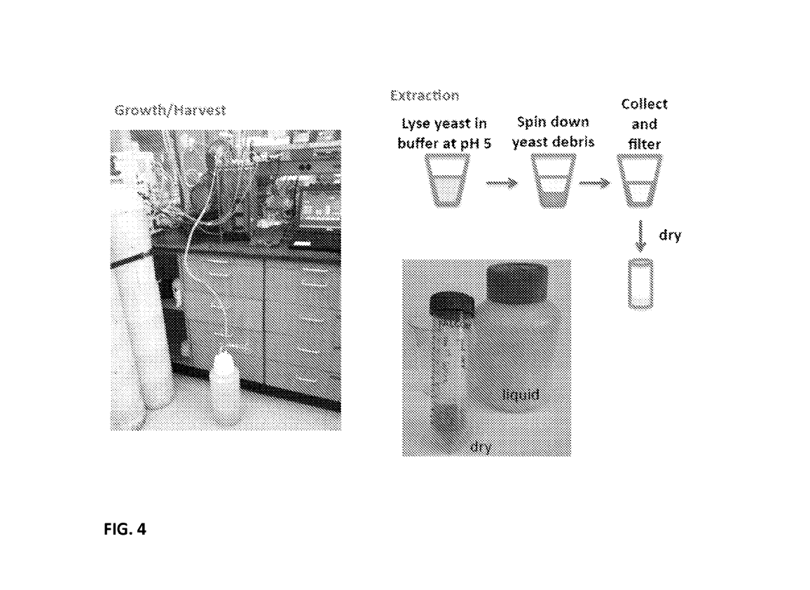

FIG. 4 shows a schematic of the process of generating yeast extract from the culture. The MATα nma1 strain overexpressing human NAMPT-tag gene was cultured in synthetic complete medium without uracil and with 2% raffinose and 2% galactose. This yeast culture was transferred to the bioreactor containing 4 L of rich medium containing 1% yeast extract, 2% peptone, 2% galactose, 2 mM nicotinamide and 0.004% adenine and allow to grow for 24 hours. Yeast cells were harvested and stored at −80 ° C. For NMN extraction, yeast cells were washed with distilled water, resuspended in 10 mM Tris buffer at pH 5.0, lysed by bead beating and centrifuge to separate the soluble and insoluble fractions. The soluble fraction was collected, filtered through a 10 kDa cutoff membrane and ready for nicotinamide mononucleotide (NMN) quantification. This extract can be used directly to raise NAD+ levels in mammalian cells such as the human dermal fibroblasts or primary fibroblasts derived from patients with mitochondrial diseases. The extract can also be lyophilized for easier storage.

### Example 2

### NMN in Cellular Extracts and Purified NMN Raise the Level of NAD⁺ in Cells

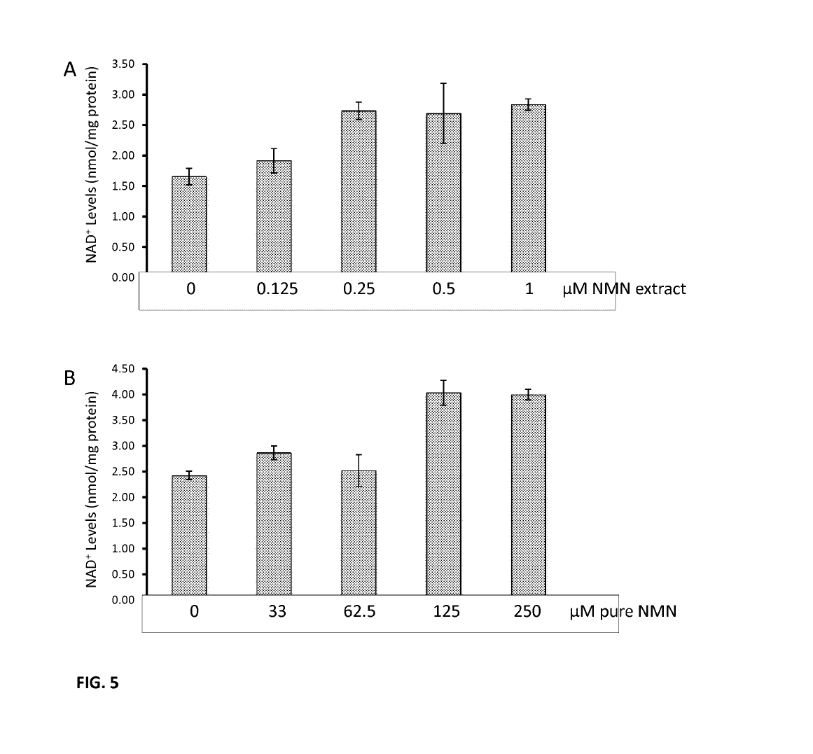

The effect of pure NMN and partially purified NMN extract on the NAD⁺ level in cells was investigated. Briefly, human dermal fibroblasts (HDFa) were treated with varying concentrations of NMN extract or pure NMN for 24 hours and NAD⁺ levels were determined. Pure NMN was obtained from Sigma. Partially purified NMN extract was obtained using the methods described above. Treatment of cells with either pure NMN or NMN extract resulted in increased levels of NAD⁺ (FIG. 5). Pure NMN increased NAD⁺ levels in cells at concentrations between 33 and 250 μM. NMN extract increased NAD⁺ levels at concentrations of 0.125 μM and 1 μM.

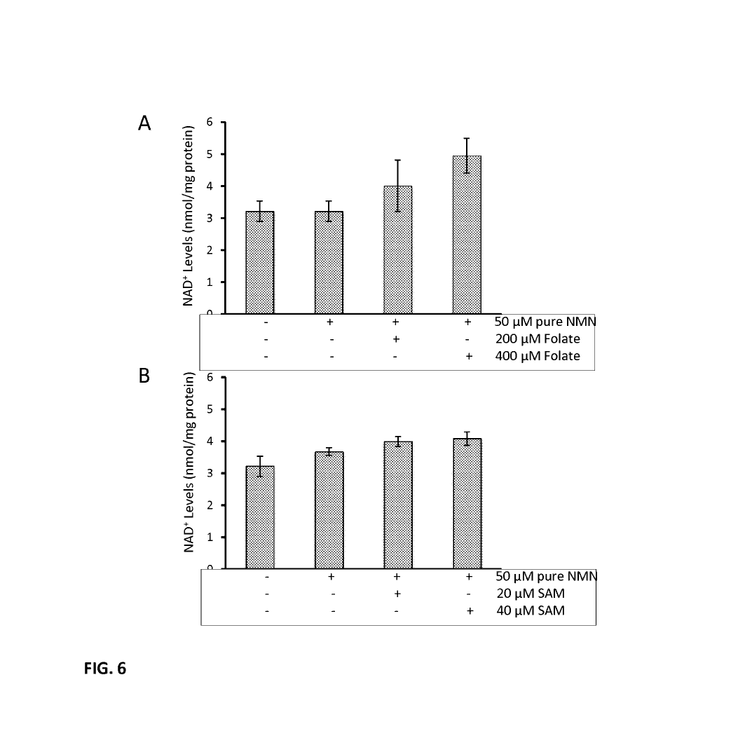

Partially purified NMN extract was analyzed and found to contain metabolites in addition to NMN, for example folate and S-adenosyl-L-methionine (SAM). As shown in FIG. 6, folate and SAM each have a synergistic effect for raising NAD⁺ levels in combination with NMN.

### REFERENCES

1. Haigis, M. C. and D. A. Sinclair, Mammalian sirtuins: biological insights and disease relevance. Annu Rev Pathol, 2010. 5: p. 253-95.
2. Rodgers, J. T., et al., Nutrient control of glucose homeostasis through a complex of PGC-1alpha and SIRT1. Nature, 2005. 434(7029): p. 113-8.
3. Gerhart-Hines, Z., et al., Metabolic control of muscle mitochondrial function and fatty acid oxidation through SIRT1/PGC-1alpha. The EMBO Journal, 2007. 26(7): p. 1913-23.
4. Baur, J. A., et al., Resveratrol improves health and survival of mice on a high-calorie diet. Nature, 2006. 444(7117): p. 337-42.
5. Lagouge, M., et al., Resveratrol improves mitochondrial function and protects against metabolic disease by activating SIRT1 and PGC-1alpha. Cell, 2006. 127(6): p. 1109-22.
6. Minor, R. K., et al., SRT1720 improves survival and healthspan of obese mice. Scientific Reports, 2011. 1: p. 70.
7. Cohen, H. Y., et al., Calorie restriction promotes mammalian cell survival by inducing the SIRT1 deacetylase. Science, 2004. 305(5682): p. 390-2.
8. Banks, A. S., et al., SirT1 gain of function increases energy efficiency and prevents diabetes in mice. Cell Metabolism, 2008. 8(4): p. 333-41.
9. Pfluger, P. T., et al., Sirt1 protects against high-fat diet-induced metabolic damage. Proceedings of the National Academy of Sciences of the United States of America, 2008. 105(28): p. 9793-8.
10. Bordone, L., et al., SIRT1 transgenic mice show phenotypes resembling calorie restriction. Aging Cell, 2007. 6(6): p. 759-67.
11. Zhang, Q. J., et al., Endothelium-specific overexpression of class III deacetylase SIRT1 decreases atherosclerosis in apolipoprotein E-deficient mice. Cardiovascular Research, 2008. 80(2): p. 191-9.
12. Oberdoerffer, P., et al., SIRT1 redistribution on chromatin promotes genomic stability but alters gene expression during aging. Cell, 2008. 135(5): p. 907-18.
13. Herranz, D., et al., Sirt1 improves healthy ageing and protects from metabolic syndrome-associated cancer. Nature Communications, 2010. 1: p. 3.
14. de Oliveira, R. M., T. F. Pais, and T. F. Outeiro, Sirtuins: common targets in aging and in neurodegeneration. Current Drug Targets, 2010. 11(10): p. 1270-80.
15. Qin, W., et al., Neuronal SIRT1 activation as a novel mechanism underlying the prevention of Alzheimer disease amyloid neuropathology by calorie restriction. The Journal of Biological Chemistry, 2006. 281(31): p. 21745-54.
16. Donmez, G., et al., SIRT1 suppresses beta-amyloid production by activating the alpha-secretase gene ADAM10. Cell, 2010. 142(2): p. 320-32.
17. Civitarese, A. E., et al., Calorie restriction increases muscle mitochondrial biogenesis in healthy humans. PLoS Medicine, 2007. 4(3): p. e76.
18. Choi, J. S., K. M. Choi, and C. K. Lee, Caloric restriction improves efficiency and capacity of the mitochondrial electron transport chain in Saccharomyces cerevisiae. Biochemical and Biophysical Research Communications, 2011. 409(2): p. 308-14.
19. Lopez-Lluch, G., et al., Calorie restriction induces mitochondrial biogenesis and bioenergetic efficiency. Proceedings of the National Academy of Sciences of the United States of America, 2006. 103(6): p. 1768-73.
20. Cerqueira, F. M., et al., Long-term intermittent feeding, but not caloric restriction, leads to redox imbalance, insulin receptor nitration, and glucose intolerance. Free Radical Biology & Medicine, 2011. 51(7): p. 1454-60.
21. Niemann, B., et al., Caloric restriction delays cardiac ageing in rats: role of mitochondria. Cardiovascular Research, 2010. 88(2): p. 267-76.
22. Hubbard, B. P., et al., Evidence for a common mechanism of SIRT1 regulation by allosteric activators. Science, March 8th, in press, 2013.
23. Abe, N., et al., Altered sirtuin deacetylase gene expression in patients with a mood disorder. J Psychiatr Res, 2011. 45(8): p. 1106-12.
24. Potente, M., et al., SIRT1 controls endothelial angiogenic functions during vascular growth. Genes Dev, 2007. 21(20): p. 2644-58.
25. Guarani, V., et al., Acetylation-dependent regulation of endothelial Notch signalling by the SIRT1 deacetylase. Nature, 2011. 473(7346): p. 234-8.
26. Hubbard, B. P., et al., Evidence for a common mechanism of SIRT1 regulation by allosteric activators. Science, 2013. 339(6124): p. 1216-9.
27. Anderson, R. M., et al., Manipulation of a nuclear NAD+ salvage pathway delays aging without altering steady-state NAD+ levels. J Biol Chem, 2002. 277(21): p. 18881-90.
28. Anderson, R. M., et al., Nicotinamide and PNC1 govern lifespan extension by calorie restriction in Saccharomyces cerevisiae. Nature, 2003. 423(6936): p. 181-5.
29. Zhang, R. Y., et al., A fluorometric assay for high-throughput screening targeting nicotinamide phosphoribosyltransferase. Anal Biochem, 2011. 412(1): p. 18-25.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A method for producing nicotinamide mononucleotide (NMN), comprising:
providing an isolated cell that overexpresses a nicotinamide phosphoribosyltransferase (Nampt) enzyme; and
culturing the isolated cell in the presence of nicotinamide (NAM).

2. The method of claim 1, wherein the isolated cell has reduced expression and/or activity of one or more of Pnc1, Std1 and Ins1 relative to a wild type cell.

3. The method of claim 1, wherein the isolated cell overexpresses a 5-phosphoribosyl-1-pyrophosphate (Prs1) enzyme.

4. The method of any one of claims claim 1 to 3, wherein the isolated cell recombinantly expresses a nucleic acid encoding the Nampt enzyme and/or a nucleic acid encoding the Prs1 enzyme.

5. The method of claim 4, wherein the isolated cell recombinantly expresses a nucleic acid encoding the Nampt enzyme and a nucleic acid encoding the Prs1 enzyme that are fused to each other.

6. The method of any one of claims 1 to 5, wherein adenine biosynthesis is disrupted in the isolated cell.

7. The method of claim 6, wherein the isolated cell has reduced expression of an adenine deaminase enzyme.

8. The method of claim 6 or 7, wherein the isolated cell is cultured in the presence of adenine.

9. The method of any one of claims 1 to 8, wherein expression of Nicotinamide Mononucleotide Adenylyltransferase 1 (NMA1) and/or Nicotinamide Mononucleotide Adenylyltransferase 2 (NMA2) is reduced in the isolated cell relative to a wild type cell.

10. The method of any one of claims 1 to 9, wherein the NMN is harvested from the isolated cell or is collected from cell culture medium produced by culturing the isolated cell.

11. The method of any one of claims 1 to 9, wherein the isolated cell is cultured in the presence of tryptophan.

12. The method of any one of claims 1 to 11, wherein the isolated cell is a fungal cell.

13. The method of claim 12, wherein the fungal cell is a yeast cell.

14. The method of claim 13, wherein the yeast cell is a Saccharomyces cell.

15. The method of any one of claims 1 to 11, wherein the isolated cell is a bacterial cell.

16. An isolated cell that recombinantly expresses a nucleic acid encoding a nicotinamide phosphoribosyltransferase (Nampt) enzyme and that has reduced expression of Pnc1 relative to a wild type cell.

17. The isolated cell of claim 16, wherein the isolated cell has reduced expression of one or both of Std1 and Ins1 relative to a wild type cell.

18. The isolated cell of claim 16 or 17, wherein the isolated cell overexpresses a 5-phosphoribosyl-1-pyrophosphate (Prs1) enzyme.

19. The isolated cell of any one of claims claim 16 to 18, wherein the isolated cell recombinantly expresses a nucleic acid encoding the Nampt enzyme and/or a nucleic acid encoding the Prs1 enzyme.

20. The isolated cell of claim 19, wherein the isolated cell recombinantly expresses a nucleic acid encoding the Nampt enzyme and a nucleic acid encoding the Prs1 enzyme that are fused to each other.

21. The isolated cell of any one of claims 16 to 20, wherein adenine biosynthesis is disrupted in the cell.

22. The isolated cell of claim 21, wherein the isolated cell has reduced expression of an adenine deaminase enzyme.

23. The isolated cell of claim 21 or 22, wherein the isolated cell is cultured in the presence of adenine.

24. The isolated cell of any one of claims 16 to 23, wherein expression of Nicotinamide Mononucleotide Adenylyltransferase 1 (NMA1) and/or Nicotinamide Mononucleotide Adenylyltransferase 2 (NMA2) is reduced in the isolated cell relative to a wild type cell.

25. The isolated cell of any one of claims 16 to 24, wherein the NMN is produced by culturing the isolated cell.

26. The isolated cell of any one of claims 16 to 25, wherein the isolated cell is a fungal cell.

27. The isolated cell of claim 26, wherein the fungal cell is a yeast cell.

28. The isolated cell of claim 27, wherein the yeast cell is a Saccharomyces cell.

29. The isolated cell of any one of claims 16 to 25, wherein the isolated cell is a bacterial cell.

30. Cell extracts produced from the isolated cell of any one of claims 16 to 29.

31. The cell extract of claim 30, further comprising folate and/or S-adenosyl-L-methionine.

32. A composition comprising the cell extracts of claim 30, wherein the composition comprises NMN.

33. The composition of claim 32, further comprising folate and/or S-adenosyl-L-methionine.

34. A cosmetic, pharmaceutical, nutraceutical or food product produced by culturing the isolated cell of any one of claims 16 to 29.

35. A cosmetic, pharmaceutical, nutraceutical or food product comprising the cell extracts of claim 30.

36. The cosmetic, pharmaceutical, nutriceutical or food product of claim 35, further comprising folate and/or S-adenosyl-L-methionine.

37. A method for producing nicotinamide mononucleotide (NMN), comprising:
providing an isolated cell that overexpresses one or more of Nicotinamide Mononucleotide Adenylyltransferase 1 (NMA1), Nicotinamide Mononucleotide Adenylyltransferase 2 (NMA2), nicotinamide mononucleotide adenylyltransferase 1 (NMNAT1), nicotinamide mononucleotide adenylyltransferase 2 (NMNAT2) and nicotinamide mononucleotide adenylyltransferase 3 (NMNAT3); and
culturing the isolated cell to produce NMN.

38. A method for producing nicotinamide mononucleotide (NMN) in vitro, comprising:
incubating NAD+ in vitro with one or more of Nicotinamide Mononucleotide Adenylyltransferase 1 (NMA1), Nicotinamide Mononucleotide Adenylyltransferase 2 (NMA2), nicotinamide mononucleotide adenylyltransferase 1 (NMNAT1), nicotinamide mononucleotide adenylyltransferase 2 (NMNAT2) and nicotinamide mononucleotide adenylyltransferase 3 (NMNAT3);
thereby producing NMN in vitro.

39. A composition comprising cell extracts from recombinant cells that produce NMN.

40. The composition of claim 39, further comprising folate and/or S-adenosyl-L-methionine.

41. The composition of claim 39 or 40, wherein the recombinant cells are yeast cells.

42. The composition of claim 39 or 40, wherein the yeast cells are Saccharomyces cells.

43. The composition of any one of claims 39 to 42, wherein the recombinant cells express a nicotinamide phosphoribosyltransferase (Nampt) enzyme.

44. A cosmetic, pharmaceutical, nutraceutical or food product comprising the composition of any one of claims 39 to 43.

45. An isolated yeast strain that expresses a nicotinamide phosphoribosyltransferase (Nampt) enzyme and that has that has reduced expression of Pnc1 relative to a wild type cell.

46. The cosmetic, pharmaceutical, nutraceutical or food product of any one of claims 34, 35 or 44, wherein the cosmetic is a cream, serum or lotion.

47. The cosmetic, pharmaceutical, nutraceutical or food product of claim 46, wherein the cream, serum or lotion contains NMN and one or more of folate and S-adenosyl-L-methionine.

48. A cosmetic, pharmaceutical, nutraceutical or food product that contains NMN and one or more of folate and S-adenosyl-L-methionine.

49. The cosmetic, pharmaceutical or nutraceutical of claim 48, wherein the cosmetic is a cream, serum or lotion.

50. The cosmetic, pharmaceutical, nutraceutical or food product of any one of claims 34, 35, 44 or 48, wherein the cosmetic or pharmaceutical is administered in an eye drop, occlusive tape or wrapping, patch, or in a formulation for systemic administration, including a pill form or a composition for injection.

51. The cosmetic, pharmaceutical, nutraceutical or food product of any one of claims 34-36, 44 and 46-50, for use in treatment or prevention of psoriasis or rheumatoid arthritis.

52. A method for treating or preventing psoriasis or rheumatoid arthritis in a subject in need thereof, comprising:
administering to the subject an effective amount of a composition comprising NMN.

53. The method of claim 52, wherein the composition further comprises folate and/or S-adenosyl-L-methionine.
